(19) United States
(12) Patent Application Publication
(10) Pub. No.: US 2012/0264927 A1
(43) Pub. Date: Oct. 18, 2012

(54) METHODS FOR LABELING GLYCANS

(76) Inventors: Ian Christopher Parsons, Belmont, MA (US); Lakshmanan Thiruneelakantapillai, Boston, MA (US); Carlos J. Bosques, Arlington, MA (US); Brian Edward Collins, Arlington, MA (US)

(21) Appl. No.: 12/595,934

(22) PCT Filed: Apr. 15, 2008
(86) PCT No.: PCT/US08/60303

§ 371 Date: Feb. 25, 2010

Related U.S. Application Data
(60) Provisional application No. 60/923,678, filed on Apr. 16, 2007.

(51) Int. Cl. C07H 1/00

(52) U.S. Cl. 536/53; 536/55; 536/55.3

(57) ABSTRACT

Methods for labeling glycans that include a step of freeze-drying a labeled glycan preparation. The labeled glycan preparation is maintained in a substantially frozen state for the duration of the freeze-drying process.

### CROSS-REFERENCE TO RELATED APPLICATIONS

This application claims priority to United States provisional application, Ser. No. 60/923,678, filed Apr. 16, 2007, the entire contents of which are incorporated herein by reference.

### BACKGROUND

Glycans have low intrinsic spectral activity and are therefore difficult to detect in their native form by standard spectroscopic techniques (e.g., by absorption or fluorescence based techniques). As a result, a variety of methods have been developed to label glycans with a detectable moiety. The most widely used labeling methods include radiolabeling (Varki, Methods Enzymol. 230:16-31, 1994) and conjugation with UV-absorbing or fluorescent probes (Hase et al., J. Biochem. 90:407-414, 1981). Labeling methods have also been developed in order to facilitate analysis of glycans by mass spectroscopy and nuclear magnetic resonance (NMR).

### SUMMARY

The present disclosure provides improved methods for processing labeled glycans. Specifically, we have shown that freeze-drying a labeled glycan preparation can significantly enhance the stability of the labeled glycan as compared to drying the preparation by other methods, e.g., by evaporation. In addition, we have found that the stability of the labeled glycan can vary depending on whether the preparation is maintained in a substantially frozen state for the duration of the freeze-drying process.

### DEFINITIONS

Approximately, About: As used herein, the term “approximately” or “about,” as applied to one or more values of interest, refers to a value that is similar to a stated reference value. In certain embodiments, the terms “approximately” or “about” refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of the stated reference value.

Biological sample: The term “biological sample”, as used herein, refers to any solid or fluid sample obtained from, excreted by or secreted by any living cell or organism, including, but not limited to, tissue culture, bioreactors, human or animal tissue, plants, fruits, vegetables, single-celled microorganisms (such as bacteria and yeasts) and multicellular organisms. For example, a biological sample can be a biological fluid obtained from, e.g., blood, plasma, serum, urine, bile, seminal fluid, cerebrospinal fluid, aqueous or vitreous humor, or any bodily secretion, a transudate, an exudate (e.g., fluid obtained from an abscess or any other site of infection or inflammation), or fluid obtained from a joint (e.g., a normal joint or a joint affected by disease such as a rheumatoid arthritis, osteoarthritis, gout or septic arthritis). A biological sample can also be, e.g., a sample obtained from any organ or tissue (including a biopsy or autopsy specimen), can comprise cells (whether primary cells or cultured cells), medium conditioned by any cell, tissue or organ, tissue culture.

Cell-surface glycoprotein: As used herein, the term “cell-surface glycoprotein” refers to a glycoprotein, at least a portion of which is present on the exterior surface of a cell. In some embodiments, a cell-surface glycoprotein is a protein that is positioned on the cell surface such that at least one of the glycan structures is present on the exterior surface of the cell.

Cell-surface glycan: A “cell-surface glycan” is a glycan that is present on the exterior surface of a cell. In many embodiments, a cell-surface glycan is covalently linked to a polypeptide as part of a cell-surface glycoprotein. A cell-surface glycan can also be linked to a cell membrane lipid.

Freeze-drying: As used herein, the term “freeze-drying” refers to a process in which a solvent is removed from a preparation by sublimation from a frozen state.

Glycan: As is known in the art and used herein “glycans” are sugars. Glycans can be monomers or polymers of sugar residues, but typically contain at least three sugars, and can be linear or branched. A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc). The term “glycan” includes homo and heteropolymers of sugar residues. The term “glycan” also encompasses a glycan component of a glycoconjugate (e.g., of a glycoprotein, glycolipid, proteoglycan, etc.). The term also encompasses free glycans, including glycans that have been cleaved or otherwise released from a glycoconjugate.

Glycan preparation: The term “glycan preparation” as used herein refers to a set of glycans obtained according to a particular production method. In some embodiments, glycan preparation refers to a set of glycans obtained from a glycoprotein preparation (see definition of glycoprotein preparation below). A “labeled glycan preparation” is a preparation that includes a labeled glycan, i.e., a glycan that has been reacted with a labeling agent.

Glycoconjugate: The term “glycoconjugate”, as used herein, encompasses all molecules in which at least one sugar moiety is covalently linked to at least one other moiety. The term specifically encompasses all biomolecules with covalently attached sugar moieties, including for example N-linked glycoproteins, O-linked glycoproteins, glycolipids, proteoglycans, etc.

Glycoform: The term “glycoform”, is used herein to refer to a particular form of a glycoconjugate. That is, when the same backbone moiety (e.g., polypeptide, lipid, etc) that is part of a glycoconjugate has the potential to be linked to different glycans or sets of glycans, then each different version of the glycoconjugate (i.e., where the backbone is linked to a particular set of glycans) is referred to as a “glycoform”.

Glycolipid: The term “glycolipid” as used herein refers to a lipid that contains one or more covalently linked sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may be comprised of one or more branched chains. In certain embodiments of the disclosure, sugar moieties may include sulfate and/or phosphate groups. In certain embodiments, glycoproteins contain O-linked sugar moieties; in certain embodiments, glycoproteins contain N-linked sugar moieties.

Glycoprotein: As used herein, the term “glycoprotein” refers to a protein that contains a peptide backbone covalently linked to one or more sugar moieties (i.e., glycans). As is understood by those skilled in the art, the peptide backbone typically comprises a linear chain of amino acid residues. In certain embodiments, the peptide backbone spans the cell membrane, such that it comprises a transmembrane portion and an extracellular portion. In certain embodiments, a peptide backbone of a glycoprotein that spans the cell membrane comprises an intracellular portion, a transmembrane portion, and an extracellular portion. In certain embodiments, methods of the present disclosure comprise cleaving a cell surface glycoprotein with a protease to liberate the extracellular portion of the glycoprotein, or a portion thereof, wherein such exposure does not substantially rupture the cell membrane. The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. In certain embodiments of the disclosure, sugar moieties may include sulfate and/or phosphate groups. Alternatively or additionally, sugar moieties may include acetyl, glycolyl, propyl or other alkyl modifications. In certain embodiments, glycoproteins contain O-linked sugar moieties; in certain embodiments, glycoproteins contain N-linked sugar moieties. In certain embodiments, methods disclosed herein comprise a step of analyzing any or all of cell surface glycoproteins, liberated fragments (e.g., glycopeptides) of cell surface glycoproteins, cell surface glycans attached to cell surface glycoproteins, peptide backbones of cell surface glycoproteins, fragments of such glycoproteins, glycans and/or peptide backbones, and combinations thereof

Glycoprotein preparation: A “glycoprotein preparation”, as that term is used herein, refers to a set of individual glycoprotein molecules, each of which comprises a polypeptide having a particular amino acid sequence (which amino acid sequence includes at least one glycosylation site) and at least one glycan covalently attached to the at least one glycosylation site. Individual molecules of a particular glycoprotein within a glycoprotein preparation typically have identical amino acid sequences but may differ in the occupancy of the at least one glycosylation sites and/or in the identity of the glycans linked to the at least one glycosylation sites. That is, a glycoprotein preparation may contain only a single glycoform of a particular glycoprotein, but more typically contains a plurality of glycoforms. Different preparations of the same glycoprotein may differ in the identity of glycoforms present (e.g., a glycoform that is present in one preparation may be absent from another) and/or in the relative amounts of different glycoforms.

Glycosidase: The term “glycosidase” as used herein refers to an agent that cleaves a covalent bond between sequential sugars in a glycan or between the sugar and the backbone moiety (e.g. between sugar and peptide backbone of glycoprotein). In some embodiments, a glycosidase is an enzyme. In certain embodiments, a glycosidase is a protein (e.g., a protein enzyme) comprising one or more polypeptide chains. In certain embodiments, a glycosidase is a chemical cleavage agent.

Glycosylation pattern: As used herein, the term “glycosylation pattern” refers to the set of glycan structures present on a particular sample. For example, a particular glycoconjugate (e.g., glycoprotein) or set of glycoconjugates (e.g., set of glycoproteins) will have a glycosylation pattern. In some embodiments, reference is made to the glycosylation pattern of cell surface glycans. A glycosylation pattern can be characterized by, for example, the identities of glycans, amounts (absolute or relative) of individual glycans or glycans of particular types, degree of occupancy of glycosylation sites, etc., or combinations of such parameters.

N-glycan: The term “N-glycan”, as used herein, refers to a polymer of sugars that has been released from a glycoconjugate but was formerly linked to the glycoconjugate via a nitrogen linkage (see definition of N-linked glycan below).

N-linked glycans: N-linked glycans are glycans that are linked to a glycoconjugate via a nitrogen linkage. A diverse assortment of N-linked glycans exists, but is typically based on the common core pentasaccharide (Man)₃(GlcNAc)(GlcNAc).

O-glycan: The term “O-glycan”, as used herein, refers to a polymer of sugars that has been released from a glycoconjugate but was formerly linked to the glycoconjugate via an oxygen linkage (see definition of O-linked glycan below).

O-linked glycans: O-linked glycans are glycans that are linked to a glycoconjugate via an oxygen linkage. O-linked glycans are typically attached to glycoproteins via N-acetyl-D-galactosamine (GalNAc) or via N-acetyl-D-glucosamine (GlcNAc) to the hydroxyl group of L-serine (Ser) or L-threonine (Thr). Some O-linked glycans also have modifications such as acetylation and sulfation. In some instances O-linked glycans are attached to glycoproteins via fucose or mannose to the hydroxyl group of L-serine (Ser) or L-threonine (Thr).

Protease: The term “protease” as used herein refers to an agent that cleaves a peptide bond between sequential amino acids in a polypeptide chain. In some embodiments, a protease is an enzyme (i.e., a proteolytic enzyme). In certain embodiments, a protease is a protein (e.g., a protein enzyme) comprising one or more polypeptide chains. In certain embodiments, a protease is a chemical cleavage agent.

Protein: In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

Substantially: As used herein, the term “substantially” refers to the qualitative condition of exhibiting total or near-total extent or degree of a characteristic or property of interest. The term “substantially” is used herein to capture the potential lack of a clear line between different phases of matter. To give but one example, when it is said that a preparation is maintained in a “substantially” frozen state for the duration of the freeze-drying process, it is meant to indicate that all or most of the preparation remains in a frozen state for the duration of the freeze-drying process. In certain embodiments, the term “substantially”, as applied to frozen preparations, refers to situations wherein 10%, 9%, 8%, 7%, 6%, 5%, 4%, 3%, 2%, 1%, or less of the preparation melts during the freeze-drying process. In certain embodiments, the term “substantially”, as applied to frozen preparations, refers to a situation wherein none of the preparation melts during the freeze-drying process.

### BRIEF DESCRIPTION OF THE DRAWING

FIG. 1 shows HPLC analysis results of an unprocessed standard labeled glycan preparation (2AB-A2F, upper graph) and the same preparation after it was allowed to evaporate (lower graph) or after it was freeze-dried but allowed to melt during freeze-drying (middle graph). In both cases significant decomposition was observed resulting in elevated levels of the free 2AB label being detected by HPLC (retention time of ˜10 minutes as opposed to ˜21 minutes for the undecomposed 2AB-A2F).

FIG. 2 shows HPLC analysis results of a mixture of 2AB-labeled glycans that were separated and fractionated by ion-exchange (IEX) chromatography (upper graph). Fractions 4, 5, 6 and 8 were collected and freeze-dried while maintaining the preparations in a substantially frozen state for the duration of the freeze-drying process. The fractions were then dissolved and analyzed by ion-exchange (IEX) chromatography (lower graphs). No change in the elution profile, and no significant release of the free 2AB label, was detected demonstrating a significant improvement over the results of FIG. 1.

FIG. 3 shows NMR analysis results of a glycan preparation before and after labeling according to a method disclosed in the Examples. Based on the ˜2% detection sensitivity of our NMR analysis, these results show that the methods are able to achieve high yields (greater than 98%).

FIG. 4 shows a representative HPLC separation of a mixture of labeled N-glycans that were labeled 2-aminobenzamide (2AB) according to the methods of the present disclosure.

FIGS. 5-6 show some representative mass spectra obtained with N-glycans labeled (with 2AB or 2AA) according to the methods of the present disclosure.

### DETAILED DESCRIPTION OF CERTAIN EMBODIMENTS

The present disclosure provides improved methods for processing labeled glycans. Specifically, we have shown that freeze-drying a labeled glycan preparation can significantly enhance the stability of the labeled glycan as compared to drying the preparation by other methods, e.g., by evaporation. In addition, we have found that the stability of the labeled glycan can vary depending on whether the preparation is maintained in a substantially frozen state for the duration of the freeze-drying process.

Freeze-Drying

In one aspect, the present disclosure provides a method in which a preparation that includes a labeled glycan is freeze-dried. In one embodiment, the preparation is maintained in a substantially frozen state for the duration of the freeze-drying step. Freeze-drying (also known as lyophilization) is a process which involves freezing the material in question and then reducing the surrounding pressure (and optionally heating the material) to allow the frozen water (or other solvent) in the material to sublime directly from the solid phase to gas. In general, the freeze-drying process involves two stages, namely freezing and drying. In certain embodiments, the drying stage is divided into primary and secondary drying phases.

The freezing stage can be done by placing the preparation in a container (e.g., a flask, eppendorf tube, etc.) and optionally rotating the container in a bath which is cooled by mechanical refrigeration (e.g., using dry ice and methanol, liquid nitrogen, etc.). In one embodiment, the freezing step involves cooling the preparation to a temperature that is below the eutectic point of the preparation. Without limitation, the eutectic point of labeled glycan preparations is typically in the range of about −10 to 10° C. depending on the nature of the solvent (e.g., aqueous, DMSO, etc.). Since the eutectic point occurs at the lowest temperature where the solid and liquid phase of the material can coexist, maintaining the material at a temperature below this point ensures that sublimation rather than evaporation will occur in subsequent steps. In one embodiment, the preparation is cooled to a temperature that is at least about 1° C. below the eutectic point of the preparation, e.g., at least about 5° C., about 10° C., or about 20° C. below the eutectic point of the preparation. In certain embodiments this will be in the range of about −30 to 9° C. depending on the nature of the solvent. It is to be understood that none of these ranges are limiting. For example, in certain embodiments, the preparation may be cooled to a temperature that is within the range of about −240 to 0° C., e.g., about −200 to 0° C., about −160 to 0° C., about −120 to 0° C., about −80 to 0° C., about −40 to 0° C., about −20 to 0° C., etc.

Larger crystals are easier to freeze dry. Thus in certain embodiments, in order to produce larger crystals the preparation can be frozen slowly (e.g., over a period of about 5 to 20 minutes) or can be cycled up and down within a temperature range. For example, in the case of labeled glycans, the temperature can be cycled anywhere between about −240 and 25° C., e.g., about −200 to 25° C., about −160 to 10° C., about −120 to 10° C., about −80 to 10 ° C., about -40 to 10° C., about −20 to 10° C., etc. for a period of time. In one embodiment, the cycling may oscillate around a gradually decreasing temperature. In one embodiment, the cycling may be followed by a gradual cooling phase. In one embodiment, the cycling ends at a temperature that is below the eutectic point of the preparation. For example the cycling may end at a temperature that is at least about 1° C. below the eutectic point of the preparation, e.g., at least about 5° C., about 10 ° C., or about 20° C. below the eutectic point of the preparation.

The drying stage (or the primary drying phase when two drying phases are used) involves reducing the pressure and optionally heating the preparation to a point where the water can sublimate. The temperature is preferably not raised above the eutectic point of the preparation. In one embodiment, the pressure within the container is reduced to between about 0.005 and 0.2 mbar, e.g., between about 0.005 and 0.05 mbar and the temperature is increased to between about −80 and −10° C., e.g., between about −40 and −20° C. In other embodiments the pressure within the container is reduced to between about 0.02 and 0.12 mbar and the temperature is increased to between about −35 and −25° C. In certain embodiments, the temperature of the container is maintained at least 25° C. below the melting point of the preparation throughout this drying phase. In other embodiments, the temperature of the container is maintained between 10 and 20° C. below the melting point of the preparation throughout this drying phase. Typically, labeled glycan preparations melt between about −10 and −5° C. under the pressures commonly used during the drying stage. This drying phase typically removes the majority of the water (or other solvent) from the preparation. It will be appreciated that the freezing and drying phases are not necessarily distinct phases but can be combined in any manner. For example, in certain embodiments, the freezing and drying phases may overlap.

A secondary drying phase can optionally be used to remove residual water (or other solvent) molecules that were adsorbed during the freezing phase. Without wishing to be bound to any theory, this phase involves raising the temperature to break any physico-chemical interactions that have formed between the water (or other solvent) molecules and the frozen preparation. For example, the temperature may be increased to between about −10 and 0° C. or even between about −5 and 0° C. In certain embodiments, the temperature of the container is maintained at least 5° C. below the melting point of the preparation throughout this secondary drying phase. In other embodiments, the temperature of the container is maintained between 5 and 15° C. below the melting point of the preparation throughout this drying phase. In certain embodiments, the pressure can also be lowered during the secondary drying phase (e.g., to within a range of 0.005 to 0.05 mbar) in order to encourage sublimation. Alternatively, in certain embodiments, the pressure can be increased during the secondary drying phase (e.g., to within a range of 0.2 to 0.5 mbar).

Once the drying stage is complete, the vacuum can be broken with an inert gas (e.g., nitrogen or helium) before the freeze-dried preparation is optionally sealed.

### Labeled Glycans

It is to be understood that the methods may be applied to any preparation that includes a labeled glycan. The glycan itself may come from any source. Some of the most commonly used labels for labeling glycans are aminated. In particular various aromatic aminated labels have been described in the art and can be used according to the present disclosure.

In one aspect of the disclosure the labeled glycans are prepared by reacting a glycan preparation with an aminated label in the presence of a reducing agent so that the aminated label reacts with glycans in the preparation by reductive amination and becomes covalently linked to the glycans. It will be appreciated that any suitable reducing agent may be used. For example, borane dimethylamine or sodium cyanoborohydride complexes may be used. In one embodiment, the reaction is performed in a solution that includes a mixture of methanol and a mild acid (e.g., acetic or citric acid). Dimethylformamide (DMF) or dimethylsufoxide (DMSO) may be used in addition to or as an alternative to methanol.

In certain embodiments it may prove advantageous to provide the original glycan preparation as a freeze-dried preparation. According to such embodiments, the freeze-dried glycan preparation can be re-suspended by adding a solution that includes the aminated label followed by addition of a solution that includes the reducing agent (when applicable). Optionally, the preparation can be dried (e.g., by simple evaporation) after addition of the aminated label and then re-suspended by addition of the solution that includes the reducing agent. It will also be appreciated that the aminated label and reducing agent (when applicable) can be mixed into a single solution which is used to re-suspend the freeze-dried glycan preparation.

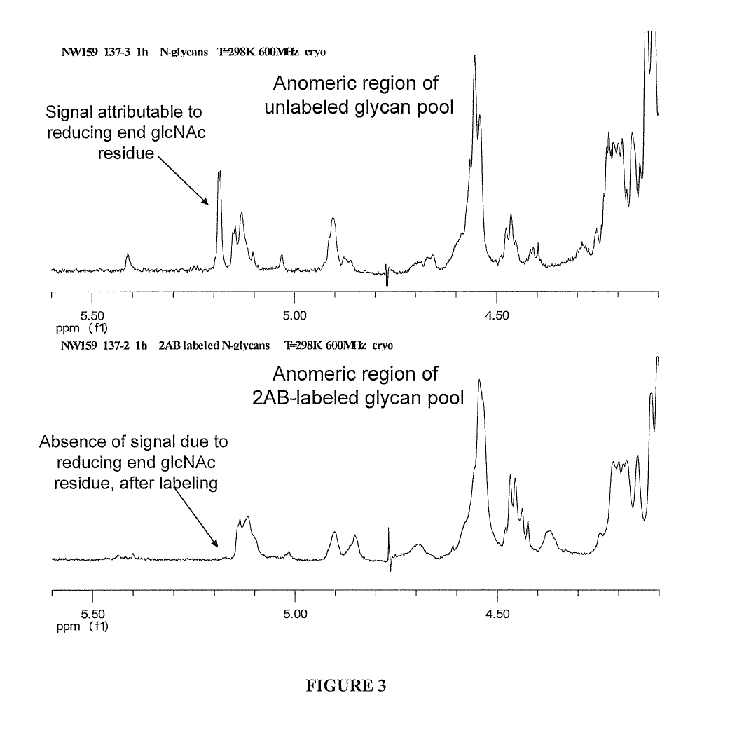

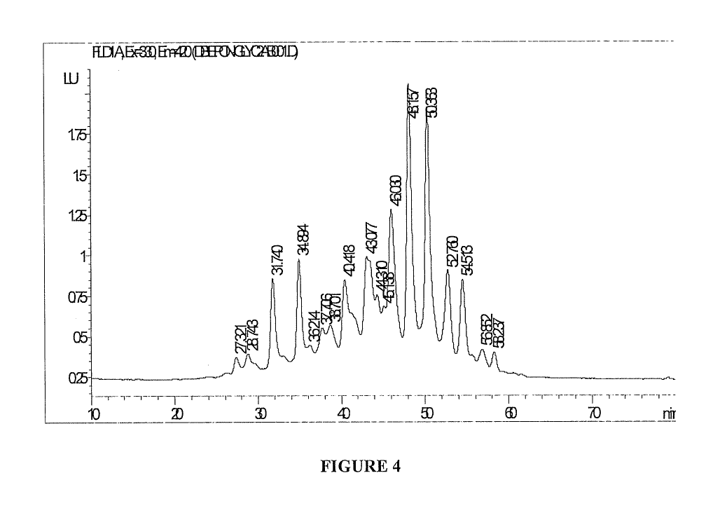

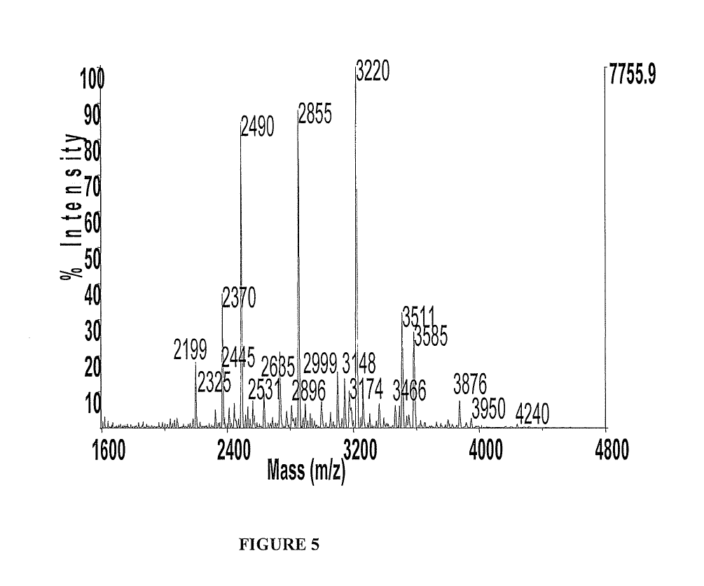

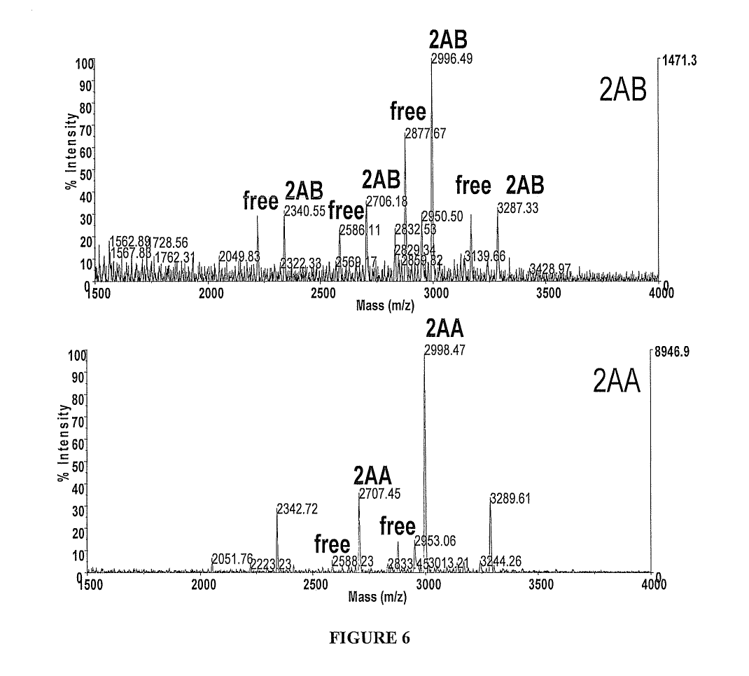

The labeling reaction can be performed at any temperature. In certain embodiments it can be performed at a temperature in the range of about 65 to 90° C. The reaction time will typically depend on the nature and concentration of the reagents and the desired yield. We have been able to achieve high yields (greater than 98% based on the ˜2% detection sensitivity of our NMR analysis) according to the methods disclosed in the Examples with reaction times on the order of 1 to 3 hours (see NMR analysis shown in FIG. 3).

In certain embodiments it may be advantageous to purify the labeled glycan preparation by removing any excess label from the preparation. This can be achieved by a variety of methods including for example paper chromatography, dialysis, etc. Optionally, the preparation may be dried by evaporation before the step of removing excess label from the preparation (e.g., using a centrifugal evaporator).

The present disclosure contemplates use of any and all known “labeling agents” for labeling of N-glycans, as provided above and herein. Additionally, the present disclosure contemplates use of any and all “labeling agents” for labeling of N-glycans, encompassed by the formula (I):

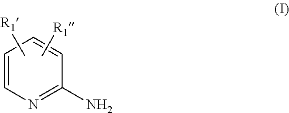

wherein

R₁′ and R₁″ are each independently —H, —NH₂, —NHR₂, —CONH₂, —COOH, —COR₃, —COOR₄, —SO₃, —SOₙR₅ where n is 1 or 2, or a substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group, or when attached to adjacent carbon atoms R₁′ and R₁″ may be taken together with the atoms to which they are attached to form a 5- to 7-membered ring optionally containing a heteroatom selected from O, N or S;

R₂, R₃, R₄ and R₅ are each independently —H or substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group; and wherein any one of the hydrogen atoms is optionally isotopically labeled as ²H or ³H; any one of the carbon atoms is optionally isotopically labeled as ¹³C; any one of the oxygen atoms is optionally isotopically labeled as ¹⁸O; any one of the nitrogen atoms is optionally isotopically labeled as ¹⁵N; and any one of the sulfur atoms is optionally isotopically labeled as ³³S or ³⁴S.

In another embodiment, a glycan can be labeled with an aminated label that is a compound of formula (II):

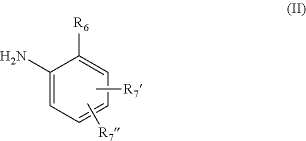

wherein:

R₆ is —H, —NH₂, —NHR₂, —CONH₂, —COOH, —COR₃, —COOR₄, —SO₃ or —SO—R₅ where n is 1 or 2;

R₂, R₃, R₄ and R₅ are each independently —H or substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group;

R₇′ and R₇″ are each independently —H, —NH₂, —NHR₂, —CONH₂, —COOH, —COR₃, —COOR₄, —SO₃, —SO—R₅ where n is 1 or 2, or a substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group, or when attached to adjacent carbon atoms R₁ and R₁′ may be taken together with the atoms to which they are attached to form a 5- to 7-membered ring optionally containing a heteroatom selected from O, N or S; and

wherein any one of the hydrogen atoms is optionally isotopically labeled as ²H or ³H; any one of the carbon atoms is optionally isotopically labeled as ¹³C; any one of the oxygen atoms is optionally isotopically labeled as ¹⁸0; any one of the nitrogen atoms is optionally isotopically labeled as ¹⁵N; and any one of the sulfur atoms is optionally isotopically labeled as ³³S or ³⁴S.

In another embodiment, the aminated label is a compound of formula (III):

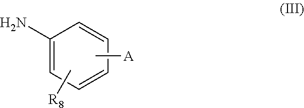

wherein

R₈ is —H, —NH₂, —NHR₂, —CONH₂, —COOH, —COR₃, —COOR₄, —SO₃ or —SOₙR₅ where n is 1 or 2;

A is a fused 5- to 15-membered substituted or unsubstituted, branched or unbranched cycloheteroalkyl, substituted or unsubstituted aryl or substituted or unsubstituted heteroaryl ring system which is optionally substituted at 1 to 5 carbon positions with —NH₂, —NHR₂, —CONH₂, —COOH, —COR₃, —COOR₄, —SO₃ or —SOₘR₅ where m is 1 or 2, or an substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group; R₂, R₃, R₄ and R₅ are each independently —H or substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group; and wherein any one of the hydrogen atoms is optionally isotopically labeled as ²H or ³H; any one of the carbon atoms is optionally isotopically labeled as ¹³C; any one of the oxygen atoms is optionally isotopically labeled as ¹⁸O; any one of the nitrogen atoms is optionally isotopically labeled as ¹⁵N; and any one of the sulfur atoms is optionally isotopically labeled as ³³S or ³⁴S.

When the glycan includes a sialic acid group then it may also be labeled by reaction with an aminated label of formula (IIA):

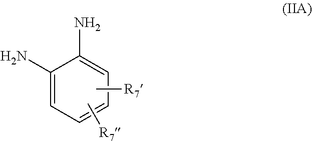

wherein

R₂, R₃, R₄ and R₅ are each independently —H or substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group;

R₇′ and R₇″ are each independently —H, —NH₂, —NHR₂, —CONH₂, —COOH, —COR₃, —COOR₄, —SO₃, —SO—R₅ where n is 1 or 2, or an substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group, or when attached to adjacent carbon atoms R₁ and R₁′ may be taken together with the atoms to which they are attached to form a 5- to 7-membered ring optionally containing a heteroatom selected from O, N or S; and

wherein any one of the hydrogen atoms is optionally isotopically labeled as ²H or ³H; any one of the carbon atoms is optionally isotopically labeled as ¹³C; any one of the oxygen atoms is optionally isotopically labeled as ¹⁸O; any one of the nitrogen atoms is optionally isotopically labeled as ¹⁵N; and any one of the sulfur atoms is optionally isotopically labeled as ³³S or ³⁴S.

It has been shown that such ortho diamines react with the sialic acid group via a condensation mechanism.

In certain embodiments, R₁′ and R₁″ are each independently —H, —NH₂, —NHR₂, —CONH₂, —COOH, —COR₃, —COOR₄, —SO₃, —SO—R₅ where n is 1 or 2, or unsubstituted, cyclic or acyclic alkyl; unsubstituted, cyclic or acyclic alkenyl; unsubstituted, cyclic or acyclic alkynyl; unsubstituted, cyclic or acyclic heteroalkyl; unsubstituted aryl, or unsubstituted heteroaryl group, or when attached to adjacent carbon atoms R₁′ and R₁″ may be taken together with the atoms to which they are attached to form a 5- to 7-membered ring optionally containing a heteroatom selected from O, N or S.

In certain embodiments, R₂, R₃, R₄ and R₅ are each independently H or unsubstituted, cyclic or acyclic alkyl; unsubstituted, cyclic or acyclic alkenyl; unsubstituted, cyclic or acyclic alkynyl; unsubstituted, cyclic or acyclic heteroalkyl, unsubstituted aryl or unsubstituted heteroaryl group.

In certain embodiments, R₇′ and R₇″ are each, independently, —H, —NH₂, —NHR₂, —CONH₂, —COOH, —COR₃, —COOR₄, —SO₃, —SOₙR₅ where n is 1 or 2, or unsubstituted, cyclic or acyclic alkyl, unsubstituted, cyclic or acyclic alkenyl, unsubstituted, cyclic or acyclic alkynyl, unsubstituted, cyclic or acyclic heteroalkyl, unsubstituted aryl or unsubstituted heteroaryl group, or when attached to adjacent carbon atoms R₁ and R₁′ may be taken together with the atoms to which they are attached to form a 5- to 7-membered ring optionally containing a heteroatom selected from O, N or S.

For example, A can be a fused 5- to 7-membered cycloheteroalkyl, aryl or heteroaryl ring system which is optionally substituted at 1 to 5 carbon positions with NH₂, NHR₂, CONH₂, COOH, COR₃, COOR₄, SO₃ or SOₘR₅ where m is 1 or 2. Alternatively, A can be a fused 6-membered heteroaryl ring system which is optionally substituted at 1 to 5 carbon positions with NH₂, NHR₂, CONH₂, COOH, COR₃, COOR₄, SO₃ or SOₘR₅ where m is 1 or 2.

Without limitation, exemplary aminated labels include 2-aminopyridine, 2,6-diaminopyridine, 2-aminobenzoic acid, 2-aminobenzamide, ortho-phenylenediamine, 6-aminoquinoline, 8-aminonaphthalene-1,3,6-trisulfonic acid, 1,2-diamino-4,5-methylenedioxy-benzene. Other specific aminated labels have been described in the art including those described in the review article by Anumula in Analytical Biochem. 350:1-23 (2006), the entire contents of which are hereby incorporated by reference.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

In general, the term “substituted” refers to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include substitution with all permissible substituents of organic compounds, any of the substituents described herein and any combination thereof that results in the formation of a stable moiety. The present disclosure contemplates any and all such combinations in order to arrive at a stable substituent/moiety. For purposes of this disclosure, heteroatoms such as nitrogen may have hydrogen substituents and/or any suitable substituent as described herein which satisfy the valencies of the heteroatoms and results in the formation of a stable moiety. The term “stable moiety,” as used herein, preferably refers to a moiety which possess stability sufficient to allow manufacture, and which maintains its integrity for a sufficient period of time to be useful for the purposes detailed herein.

The term “alkyl,” as used herein, refers to saturated, cyclic or acyclic, branched or unbranched, substituted or unsubstituted hydrocarbon radicals derived from a hydrocarbon moiety containing between one and twenty carbon atoms by removal of a single hydrogen atom. In some embodiments, the alkyl group employed contains 1-20 carbon atoms. In another embodiment, the alkyl group employed contains 1-15 carbon atoms. In another embodiment, the alkyl group employed contains 1-10 carbon atoms. In another embodiment, the alkyl group employed contains 1-8 carbon atoms. In another embodiment, the alkyl group employed contains 1-5 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more sustitutents. Alkyl group substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, alkyloxy, aryloxy, alkyloxyalkyl, azido, oxo, cyano, halo, isocyano, nitro, nitroso, azo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; haloalkyl, alkoxyalkyl, substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “cycloalkyl” refers to a cyclic alkyl group, as defined herein. Cycloalkyl groups include cyclopropyl, cyclobutyl, cyclopentyl, cyclhexyl, cycloheptyl, cyclooctyl, cyclononyl, cyclodecyl, cycloundecyl, cyclododecyl, and the like, which may bear one or more sustitutents. Cycloalkyl group substituents include, but are not limited to, any of the substituents described herein, that result in the formation of a stable moiety (e.g., e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, alkyloxy, aryloxy, alkyloxyalkyl, azido, cyano, halo, isocyano, nitro, nitroso, azo, oxo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “alkenyl,” as used herein, denotes a monovalent group derived from a cyclic or acyclic, branched or unbranched, substituted or unsubstituted hydrocarbon moiety having at least one carbon-carbon double bond by the removal of a single hydrogen atom. In certain embodiments, the alkenyl group employed contains 2-20 carbon atoms. In some embodiments, the alkenyl group contains 2-15 carbon atoms. In another embodiment, the alkenyl group employed contains 2-10 carbon atoms. In still other embodiments, the alkenyl group contains 2-8 carbon atoms. In yet another embodiments, the alkenyl group contains 2-5 carbons. Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, haloalkyl, alkyloxy, aryloxy, alkyloxyalkyl, azido, cyano, halo, isocyano, nitro, nitroso, azo, oxo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “alkynyl,” as used herein, refers to a monovalent group derived from a cyclic or acyclic, branched or unbranched, substituted or unsubstituted hydrocarbon having at least one carbon-carbon triple bond by the removal of a single hydrogen atom. In certain embodiments, the alkynyl group contains 2-20 carbon atoms. In some embodiments, the alkynyl group contains 2-15 carbon atoms. In another embodiment, the alkynyl group employed contains 2-10 carbon atoms. In still other embodiments, the alkynyl group contains 2-8 carbon atoms. In still other embodiments, the alkynyl group contains 2-5 carbon atoms. Representative alkynyl groups include, but are not limited to, ethynyl, 2-propynyl (propargyl), 1-propynyl, and the like, which may bear one or more substituents. Alkynyl group substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, alkyloxy, aryloxy, alkyloxyalkyl, azido, cyano, halo, isocyano, nitro, nitroso, azo, oxo, —CONH₂, —COOH, —COR₃, —COOR₄, —SO₃, —SOₙR₅ , wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “heteroalkyl,” as used herein, refers to an alkyl moiety, as defined herein, which includes saturated, cyclic or acyclic, branched or unbranched, substituted or unsubstituted hydrocarbon radicals, which contain one or more oxygen, sulfur, nitrogen, phosphorus, or silicon atoms, e.g., in place of carbon atoms. In certain embodiments, hetereoalkyl moieties are substituted by independent replacement of one or more of the hydrogen atoms thereon with one or more substituents. Heteroalkyl substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, alkyloxy, aryloxy, alkyloxyalkyl, azido, cyano, halo, isocyano, nitro, nitroso, azo, oxo, —CONH₂, —COOH, —COR₃, —COOR₄, —SO₃, —SOₙR₅ wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

As used herein the term “haloalkyl” designates a CₙH₂ₙ₊₁ group having from one to 2n+1 halogen atoms which may be the same or different. Examples of haloalkyl groups include CF₃, CH₂Cl, C₂H₃BrCl, C₃H₅F₂, or the like. Similarly, the term haloalkoxy designates an OCₙH₂ₙ₊₁ group having from one to 2n+1 halogen atoms which may be the same or different.

The term “alkoxyalkyl”, as used herein, refers to an alkyl group as hereinbefore defined substituted with at least one alkyloxy group.

The term “cycloheteroalkyl,” as used herein, refers to a cyclic heteroalkyl group as defined herein. A cycloheteroalkyl group refers to a fully saturated 3- to 10-membered ring system, which includes single rings of 3 to 8 atoms in size. These cycloheteroalkyl rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term cycloheteroalkyl refers to a 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Examples of cycloheteroalkyl ring systems included in the term as designated herein are the following rings wherein X₁ is NR′, O or S, and R′ is H or an optional substituent as defined herein:

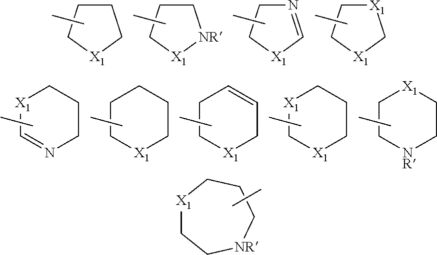

Exemplary cycloheteroalkyls include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, alkyloxy, aryloxy, alkyloxyalkyl, azido, cyano, halo, isocyano, nitro, nitroso, azo, oxo, —CONH₂, —COOH, —COR₃, —COOR₄, —SO₃, —SOₙR₅ , wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group)

The term “aryl,” as used herein, refer to stable aromatic mono- or polycyclic ring system having 3-20 ring atoms, of which all the ring atoms are carbon, and which may be substituted or unsubstituted. In certain embodiments, “aryl” refers to a mono, bi, or tricyclic C₄-C₂₀ aromatic ring system having one, two, or three aromatic rings which include, but not limited to, phenyl, biphenyl, naphthyl, and the like, which may bear one or more substituents. Aryl substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, alkyloxy, aryloxy, alkyloxyalkyl, azido, cyano, halo, isocyano, nitro, nitroso, azo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “heteroaryl,” as used herein, refer to stable aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Exemplary heteroaryls include, but are not limited to pyrrolyl, pyrazolyl, imidazolyl, pyridinyl, pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, alkyloxy, aryloxy, alkyloxyalkyl, azido, cyano, halo, isocyano, nitro, nitroso, azo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group)

The term “amino,” as used herein, refers to a group of the formula (—NH₂). A “substituted amino” refers either to a mono-substituted amino (—NHR^{h}) or a di-substituted amino (—NR^{h}₂), wherein the R^{h} substituent is any substitutent as described herein that results in the formation of a stable moiety (e.g., a cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, haloalkyl, alkyloxy, aryloxy, alkyloxyalkyl, azido, cyano, halo, oxo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group). In certain embodiments, the R^{h} substituents of the di-substituted amino group(—NR^{h}₂) form an optionally substituted 5- to 6-membered cycloheteroalkyl ring. A dialkylamino group is a di-substituted amino group, as defined herein, wherein each R^{h} is, independently, an alkyl group, or two R^{h} alkyl groups are joined together to form a 5- to 6-membered ring. Exemplary dialkylamino groups include dimethylamino, di-ethylamino, di-propylamino, di-isopropylamino, ethylisopropylamino, pyrrolidinyl, piperidinyl, and the like.

The term “hydroxy,” or “hydroxyl,” as used herein, refers to a group of the formula (—OH). A “substituted hydroxyl” refers to a group of the formula (—ORⁱ wherein R′ can be any substitutent which results in a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group)

The term “thio” or “thiol” as used herein, refers to a group of the formula (—SH). A “substituted thiol” refers to a group of the formula (—SR^{r}), wherein R^{r} can be any substitutent which results in a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “alkyloxy” refers to a “substituted hydroxyl” of the formula (—ORⁱ), wherein Rⁱ is an optionally substituted alkyl group, as defined herein, and the oxygen moiety is directly attached to the parent molecule. The term “alkylthioxy” refers to a “substituted thiol” of the formula (—SR^{r}), wherein Rr is an optionally substituted alkyl group, as defined herein, and the sulfur moiety is directly attached to the parent molecule. The term “alkylamino” refers to a “substituted amino” of the formula (—NR^{h}₂), wherein R^{h} is, independently, a hydrogen or an optionally subsituted alkyl group, as defined herein, and the nitrogen moiety is directly attached to the parent molecule.

The term “aryloxy” refers to a “substituted hydroxyl” of the formula (—ORⁱ), wherein R′ is an optionally substituted aryl group, as defined herein, and the oxygen moiety is directly attached to the parent molecule. The term “arylamino,” refers to a “substituted amino” of the formula (—NR^{h}₂), wherein R^{h} is, independently, a hydrogen or an optionally substituted aryl group, as defined herein, and the nitrogen moiety is directly attached to the parent molecule. The term “arylthioxy” refers to a “substituted thiol” of the formula (—SR^{r}), wherein R^{r} is an optionally substituted aryl group, as defined herein, and the sulfur moiety is directly attached to the parent molecule.

The term “alkyloxyalkyl” or “alkoxyalkyl” as used herein refers to an alkyloxy group, as defined herein, attached to an alkyl group attached to the parent molecule.

The term “azido,” as used herein, refers to a group of the formula (—N₃).

The term “cyano,” as used herein, refers to a group of the formula (—CN).

The terms “halo” and “halogen” as used herein refer to an atom selected from fluorine (fluoro, —F), chlorine (chloro, —Cl), bromine (bromo, —Br), and iodine (iodo, —I).

The term “isocyano,” as used herein, refers to a group of the formula (—NC).

The term “nitro,” as used herein, refers to a group of the formula (—NO₂).

The term “nitroso,” as used herein, refers to a group of the formula (—N═O).

The term “azo,” as used herein, refers to a group of the formula (—N₂).

The term “oxo,” as used herein, refers to a group of the formula (═O).

### Applications

It will be appreciated that the techniques described herein can be utilized in any of a variety of applications. In general, these techniques are useful in any application that involves the characterization of glycans. Techniques of the present disclosure may be particularly useful in facilitating applications that require glycans to be detected.

Methods in accordance with the invention can be applied to glycans obtained from a wide variety of sources including, but not limited to, therapeutic formulations (e.g., erythropoietin, insulin, human growth hormone, etc.), commercial biological products (e.g., those presented in a table below), and biological samples. A biological sample may undergo one or more analysis and/or purification steps prior to or after being analyzed according to the present invention. To give but a few examples, in some embodiments, a biological sample is treated with one or more proteases and/or exoglycosidases (e.g., so that glycans are released); in some embodiments, glycans in a biological sample are labeled with one or more detectable markers or other agents that may facilitate analysis by, for example, mass spectrometry or NMR. Any of a variety of separation and/or isolation steps may be applied to a biological sample in accordance with the present invention.

The methods can be utilized to analyze glycans in any of a variety of states including, for instance, free glycans; glycoconjugates (e.g., glycopeptides, glycolipids, proteoglycans, etc.); cell-associated glycans (e.g., nucleus-, cytoplasm-, cell membrane-associated glycans, etc.); glycans associated with cellular, extracellular, intracellular, and/or subcellular components (e.g., proteins); glycans in extracellular space (e.g., cell culture medium) etc.

Methods of the present invention may be used in one or more stages of process development for the production of a therapeutic or other commercially relevant glycoprotein of interest. Non-limiting examples of such process development stages that can employ methods of the present invention include cell selection, clonal selection, media optimization, culture conditions, process conditions, and/or purification procedure. Those of ordinary skill in the art will be aware of other process development stages.

The present disclosure can also facilitate analytical methods that monitor the extent and/or type of glycosylation occurring in a particular cell culture, thereby allowing adjustment or possibly termination of the culture in order, for example, to achieve a particular desired glycosylation pattern or to avoid development of a particular undesired glycosylation pattern.

The present disclosure can also facilitate analytical methods that assess glycosylation characteristics of cells or cell lines that are being considered for production of a particular desired glycoprotein (for example, even before the cells or cell lines have been engineered to produce the glycoprotein, or to produce the glycoprotein at a commercially relevant level).

In some embodiments of the disclosure, a desired glycosylation pattern for a particular target glycoprotein (e.g., a cell surface glycoprotein) is known, and the technology described herein allows monitoring of culture samples to assess progress of the production along a route known to produce the desired glycosylation pattern. For example, where the target glycoprotein is a therapeutic glycoprotein, for example having undergone regulatory review in one or more countries, it will often be desirable to monitor cultures to assess the likelihood that they will generate a product with a glycosylation pattern as close to the established glycosylation pattern of the pharmaceutical product as possible, whether or not it is being produced by exactly the same route. As used herein, “close” refers to a glycosylation pattern having at least about a 75%, 80%, 85%, 90%, 95%, 98%, or 99% correlation to the established glycosylation pattern of the pharmaceutical product. In such embodiments, samples of the production culture are typically taken at multiple time points and are compared with an established standard or with a control culture in order to assess relative glycosylation.

In some embodiments of the present disclosure, a desired glycosylation pattern will be more extensive. For example, in some embodiments, a desired glycosylation pattern shows high (e.g., greater than about 60%, 65%, 70%, 75%, 80%, 85%, 90%, 95% or more) occupancy of glycosylation sites; in some embodiments, a desired glycosylation pattern shows, a high degree of branching (e.g., greater than about 60%, 65%, 70%, 75%, 80%, 85%, 90%, 95% or more have tri or tetraantennary structures).

In some embodiments of the present disclosure, a desired glycosylation pattern will be less extensive. For example, in some embodiments, a desired glycosylation pattern shows low (e.g., less than about 35%, 30%, 25%, 20%, 15% or less) occupancy of glycosylation sites; and/or a low degree of branching (e.g., less than about 20%, 15%, 10%, 5%, or less have tri or tetraantennary structures).

In some embodiments, a desired glycosylation pattern will be more extensive in some aspects and less extensive than others. For example, it may be desirable to employ a cell line that tends to produce glycoproteins with long, unbranched oligosaccharide chains. Alternatively, it may be desirable to employ a cell line that tends to produce glycoproteins with short, highly branched oligosaccharide chains.

In some embodiments, a desired glycosylation pattern will be enriched for a particular type of glycan structure. For example, in some embodiments, a desired glycosylation pattern will have low levels (e.g., less than about 20%, 15%, 10%, 5%, or less) of high mannose or hybrid structures, high (e.g., more than about 60%, 65%, 70%, 75%, 80%, 85%, 90% or more) levels of high mannose structures, or high (e.g., more than about 60%, 65%, 70%, 75%, 80%, 85%, 90% or more; for example at least one per glycoprotein) or low (e.g., less than about 20%, 15%, 10%, 5%, or less) levels of phosphorylated high mannose.

In some embodiments, a desired glycosylation pattern will include at least about one sialic acid. In some embodiments, a desired glycosylation pattern will include a high (e.g., greater than about 60%, 65%, 70%, 75%, 80%, 85%, 90%, 95% or more) level of termini that are sialylated. In some embodiments, a desired glycosylation pattern that includes sialylation will show at least about 85%, 90%, 95% or more N-acetylneuraminic acid and/or less than about 15%, 10%, 5% or less N-glycolylneuraminic acid.

In some embodiments, a desired glycosylation pattern shows specificity of branch elongation (e.g., greater than about 50%, 55%, 60%, 65%, 70% or more of extension is on α1,6 mannose branches, or greater than about 50%, 55%, 60%, 65%, 70% or more of extension is on α1,3 mannose branches).

In some embodiments, a desired glycosylation pattern will include a low (e.g., less than about 20%, 15%, 10%, 5%, or less) or high (e.g., more than about 60%, 65%, 70%, 75%, 80%, 85%, 90%, 95%, or more) level of core fucosylation.

Whether or not monitoring production of a particular target protein for quality control purposes, the methods may be utilized, for example, to facilitate the monitoring of glycosylation at particular stages of development, or under particular growth conditions.

In some particular embodiments, methods can be used to facilitate the characterization and/or control or comparison of the quality of therapeutic products. To give but one example, methodologies can be used to assess glycosylation in cells producing a therapeutic protein product. Particularly given that glycosylation can often affect the activity, bioavailability, or other characteristics of a therapeutic protein product, methods for assessing cellular glycosylation during production of such a therapeutic protein product are particularly desirable. Among other things, the present methods can facilitate real time analysis of glycosylation in production systems for therapeutic proteins.

Representative therapeutic glycoprotein products whose production and/or quality can be monitored in accordance with the present disclosure include, for example, any of a variety of hematologic agents (including, for instance, erythropoietins, blood-clotting factors, etc.), interferons, colony stimulating factors, antibodies, enzymes, and hormones.

Representative commercially available glycoprotein products include, for example:

| Protein Product | Reference Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| laronidase | Aldurazyme ® |
| interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion protein | Amevive ® |
| LFA3-Ig | |
| bivalirudin | Angiomax ® |
| darbepoetin alfa | Aranesp ™ |
| bevacizumab | Avastin ™ |
| interferon beta-1a; recombinant | Avonex ® |
| coagulation factor IX | BeneFix ™ |
| Interferon beta-1b | Betaseron ® |
| Tositumomab | Bexxar ® |
| antihemophilic factor | Bioclate ™ |
| human growth hormone | BioTropin ™ |
| botulinum toxin type A | Botox ® |
| alemtuzumab | Campath ® |
| acritumomab; technetium-99 labeled | CEA-Scan ® |
| alglucerase; modified form of beta- | Ceredase ® |
| glucocerebrosidase | |
| imiglucerase; recombinant form of beta- | Cerezyme ® |
| glucocerebrosidase | |
| crotalidae polyvalent immune Fab, ovine | CroFab ™ |
| digoxin immune Fab, ovine | DigiFab ™ |
| rasburicase | Elitek ® |
| etanercept | Enbrel ® |
| epoietin alfa | Epogen ® |
| cetuximab | Erbitux ™ |
| algasidase beta | Fabrazyme ® |
| urofollitropin | Fertinex ™ |
| follitropin beta | Follistim ™ |
| teriparatide | Forteo ® |
| human somatropin | GenoTropin ® |
| glucagon | GlucaGen ® |
| follitropin alfa | Gonal-F ® |
| antihemophilic factor | Helixate ® |
| Antihemophilic Factor; Factor XIII | Hemofil ® |
| insulin | Humalog ® |
| antihemophilic factor/von Willebrand factor | Humate-P ® |
| complex-human | |
| somatotropin | Humatrope ® |
| adalimumab | HUMIRA ™ |
| human insulin | Humulin ® |
| recombinant human hyaluronidase | Hylenex ™ |
| interferon alfacon-1 | Infergen ® |
| Eptifibatide | Integrilin ™ |
| alpha-interferon | Intron A ® |
| palifermin | Kepivance |
| anakinra | Kineret ™ |
| antihemophilic factor | Kogenate ® FS |
| insulin glargine | Lantus ® |
| granulocyte macrophage colony-stimulating | Leukine ®/Leukine ® |
| factor | Liquid |
| lutropin alfa, for injection | Luveris |
| OspA lipoprotein | LYMErix ™ |
| ranibizumab | Lucentis ® |
| gemtuzumab ozogamicin | Mylotarg ™ |
| galsulfase | Naglazyme ™ |
| nesiritide | Natrecor ® |
| pegfilgrastim | Neulasta ™ |
| oprelvekin | Neumega ® |
| filgrastim | Neupogen ® |
| fanolesomab | NeutroSpec ™ |
| | (formerly LeuTech ®) |
| somatropin [rDNA] | Norditropin ®/ |
| | Norditropin Nordiflex ® |
| insulin; zinc suspension; | Novolin L ® |
| insulin; isophane suspension | Novolin N ® |
| insulin, regular; | Novolin R ® |
| insulin | Novolin ® |
| coagulation factor VIIa | NovoSeven ® |
| somatropin | Nutropin ® |
| immunoglobulin intravenous | Octagam ® |
| PEG-L-asparaginase | Oncaspar ® |
| abatacept, fully human soluable fusion protein | Orencia ™ |
| muromomab-CD3 | Orthoclone OKT3 ® |
| human chorionic gonadotropin | Ovidrel ® |
| peginterferon alfa-2a | Pegasys ® |
| pegylated version of interferon alfa-2b | PEG-Intron ™ |
| Abarelix (injectable suspension); gonadotropin- | Plenaxis ™ |
| releasing hormone antagonist | |
| epoietin alfa | Procrit ® |
| aldesleukin | Proleukin, IL-2 ® |
| somatrem | Protropin ® |
| dornase alfa | Pulmozyme ® |
| Efalizumab; selective, reversible T-cell blocker | Raptiva ™ |
| combination of ribavirin and alpha interferon | Rebetron ™ |
| Interferon beta 1a | Rebif ® |
| antihemophilic factor | Recombinate ® |
| rAHF/ntihemophilic factor | ReFacto ® |
| lepirudin | Refludan ® |
| infliximab | Remicade ® |
| abciximab | ReoPro ™ |
| reteplase | Retavase ™ |
| rituximab | Rituxan ™ |
| interferon alfa-2a | Roferon-A ® |
| somatropin | Saizen ® |
| synthetic porcine secretin | SecreFlo ™ |
| basiliximab | Simulect ® |
| eculizumab | Soliris ® |
| pegvisomant | Somavert ® |
| Palivizumab; recombinantly produced, | Synagis ™ |
| humanized mAb | |
| thyrotropin alfa | Thyrogen ® |
| tenecteplase | TNKase ™ |
| natalizumab | Tysabri ® |
| human immune globulin intravenous 5% and | Venoglobulin-S ® |
| 10% solutions | |
| interferon alfa-n1, lymphoblastoid | Wellferon ® |
| drotrecogin alfa | Xigris ™ |
| Omalizumab; recombinant DNA-derived | Xolair ® |
| humanized monoclonal antibody targeting | |
| immunoglobulin-E | |
| daclizumab | Zenapax ® |
| ibritumomab tiuxetan | Zevalin ™ |
| Somatotropin | Zorbtive ™ |
| | (Serostim ®) |

In some embodiments, the present disclosure provides methods in which glycans from different sources or samples are compared with one another. In certain embodiments, the disclosure provides methods used to monitor the extent and/or type of glycosylation occuring in different cell cultures. In some such examples, multiple samples from the same source are obtained over time, so that changes in glycosylation patterns (and particularly in cell surface glycosylation patterns) are monitored. In some embodiments, one of the samples is a historical sample or a record of a historical sample. In some embodiments, one of the samples is a reference sample. For example, in certain embodiments, methods are provided herein which can be used to monitor the extent and/or type of glycosylation occurring in different cell cultures.

In some embodiments, glycans from different cell culture samples prepared under conditions that differ in one or more selected parameters (e.g., cell type, culture type [e.g., continuous feed vs batch feed, etc.], culture conditions [e.g., type of media, presence or concentration of particular component of particular medium(a), osmolarity, pH, temperature, timing or degree of shift in one or more components such as osmolarity, pH, temperature, etc.], culture time, isolation steps, etc.) but are otherwise identical, are compared, so that effects of the selected parameter(s) on glycosylation patterns are determined. In certain embodiments, glycans from different cell culture samples prepared under conditions that differ in a single selected parameter are compared so that effect of the single selected parameter on glycosylation patterns is determined. Among other applications, therefore, use of techniques as described herein may facilitate determination of the effects of particular parameters on glycosylation patterns in cells.

In some embodiments, glycans from different batches of a glycoprotein of interest (e.g., a therapeutic glycoprotein), whether prepared by the same method or by different methods, and whether prepared simultaneously or separately, are compared. In such embodiments, the methods facilitate quality control of glycoprotein preparation. Alternatively or additionally, some such embodiments facilitate monitoring of progress of a particular culture producing a glycoprotein of interest (e.g., when samples are removed from the culture at different time points and are analyzed and compared to one another). In any of these embodiments, features of the glycan analysis can be recorded, for example in a quality control record. As indicated above, in some embodiments, a comparison is with a historical record of a prior or standard batch and/or with a reference sample of glycoprotein. In some embodiments, a comparison is with a reference glycoprotein sample.

In certain embodiments, the methods may be utilized in studies to modify the glycosylation characteristics of a cell, for example to establish a cell line and/or culture conditions with one or more desirable glycosylation characteristics. Such a cell line and/or culture conditions can then be utilized, if desired, for production of a particular target glycoconjugate (e.g., glycoprotein) for which such glycosylation characteristic(s) is/are expected to be beneficial.

In certain embodiments, techniques of the present disclosure are applied to glycans that are present on the surface of cells. In some such embodiments, the analyzed glycans are substantially free of non-cell-surface glycans. In some such embodiments, the analyzed glycans, when present on the cell surface, are present in the context of one or more cell surface glycoconjugates (e.g., glycoproteins or glycolipids).

In some particular embodiments, cell surface glycans are analyzed in order to assess glycosylation of one or more target glycoproteins of interest, particularly where such target glycoproteins are not cell surface glycoproteins. Such embodiments can allow one to monitor glycosylation of a target glycoprotein without isolating the glycoprotein itself. In certain embodiments, the present disclosure provides methods of using cell-surface glycans as a readout of or proxy for glycan structures on an expressed glycoprotein of interest. In certain embodiments, such methods include, but are not limited to, post process, batch, screening or “in line” measurements of product quality. Such methods can provide for an independent measure of the glycosylation pattern of a produced glycoprotein of interest using a byproduct of the production reaction (e.g., the cells) without requiring the use of destruction of any produced glycoprotein. According to such embodiment, methods of the present disclosure can be used to avoid the effort required for isolation of product and the potential selection of product glycoforms that may occur during isolation.

In certain embodiments, techniques of the present disclosure are applied to glycans that are secreted from cells. In some such embodiments, the analyzed glycans are produced by cells in the context of a glycoconjugate (e.g., a glycoprotein or glycolipid).

Techniques described herein can be used to facilitate the detection of desirable or undesirable glycans, for example to detect or quantify the presence of one or more contaminants in a product, or to detect or quantify the presence of one or more active or desired species.

In various embodiments the methods can be used to facilitate the detection of biomarkers that are indicative of, e.g., a disease state, prior to the appearance of symptoms and/or progression of the disease state to an untreatable or less treatable condition, by detecting one or more specific glycans whose presence or level (whether absolute or relative) may be correlated with a particular disease state (including susceptibility to a particular disease) and/or the change in the concentration of such glycans over time.

In some embodiments, techniques described herein may be combined with one or more other technologies for the detection, analysis, and or isolation of glycans or glycoconjugates.

Thus, in certain embodiments, the methods comprise releasing glycans from a glycoconjugate or cell surface to provide a glycan preparation. In certain embodiments, the glycan preparation is provided via cleavage of glycans from a glycoprotein after the cell surface glycoproteins have been liberated from the cell (e.g., through treatment with one or more proteases and/or glycosidases). In certain embodiments, the glycan preparation is provided via cleavage of glycans from cell surface glycoproteins that have not been liberated from the cell. Glycans may be released (e.g., separated, cleaved, hydrolyzed) using a variety of chemical or enzymatic methods; see generally, Kamerling, Pure Appl. Chem. (1994) 66:2235-2238; Kamerling and Vliegnenthart, in: Clinical Biochemistry, Principles, Methods, Applications, Volume 1 (A. N. Lawson, ed), Walter De Gruyter, Berlin (1989) pp. 175-263; and Allen and Kisailus, eds., Glycoconguates, Marcel Dekker Inc., New York, 1992.

Any of a variety of glycosidases that cleave glycan structures from glycoproteins, or cell surface glycoproteins, may be used in accordance with the present disclosure. Several examples of such glycosidases are reviewed in R. A. O'Neill, Enzymatic release of oligosaccharides from glycoproteins for chromatographic and electrophoretic analysis, J. Chromatogr. A 720, 201-215. 1996; and S. Prime, et al., Oligosaccharide sequencing based on exo- and endo-glycosidase digestion and liquid chromatographic analysis of the products, J. Chromatogr. A 720, 263-274, 1996. In certain embodiments, the enzyme PNGase F (Peptide N-Glycosidase F) is used to remove glycans from a glycoprotein. PNGase F is an amidase that cleaves the amide bond between the innermost GlcNAc and asparagine residues of high mannose, hybrid, and complex oligosaccharides from N-linked glycoproteins. Other suitable enzymes that can be used to cleave glycan structures from glycoproteins in accordance with the present disclosure include, but are not limited to, PNGase A and endoglycosidases (Endo-H). Those of ordinary skill in the art will be aware of other suitable enzymes for cleavage of glycans from glycoproteins. In certain embodiments, a plurality of enzymes is used to cleave glycan structures from a glycoprotein.

To improve the accessibility of the glycosylation site to the enzyme, most glycoproteins require a protein denaturation step. Typically, this is accomplished by using detergents and disulfide-reducing agents, although methods of denaturing a glycoprotein for use in accordance with the present disclosure are not limited to the use of such agents. For example, exposure to high temperature can be sufficient to denature a glycoprotein such that a suitable enzyme for cleaving glycan structures is able to access the cleavage site. In certain embodiments, a combination of detergents, disulfide-reducing agents, high temperature, and/or other agents or reaction conditions is employed to denature a glycoprotein. It is noted that glycans located at conserved Fc sites in immunoglobulin G (IgG) are easily cleaved by PNGase F. Thus, a protein denaturation step is typically not required for IgG molecules. PNGase F is also capable of removing glycans in dilute ammonium hydroxide solution. Thus, use of PNGase F to cleave glycans from glycoproteins has the advantage that the dilute ammonium hydroxide may additionally aid in solubility and some unfolding of the protein substrates.

Additionally, glycans may be cleaved from a glycoprotein using chemical methods. For example, a glycan may be released via treatment with hydrazine to provide a hydrazide of the glycan (i.e., hydrazinolysis).

Additionally, following cleavage of the glycans from the glycoprotein or cell-surface glycoprotein, the glycans may be purified to remove non-carbohydrate contaminants, such as salts, chemicals, and detergents used in enzymatic digests. The methods of purification may include, but are not limited to, the use of C18 and graphitized carbon cartridges and spin columns. In other embodiments, the method of purification may include a step of acetone precipitation of proteinaceous material from an ice-cold aqueous solution containing both proteins and glycans.

Finally, it will be appreciated that once the labeled glycans have been prepared according to the methods described herein they may be further analyzed by any technique. For example, the labeled glycans may be analyzed by chromatographic methods, mass spectrometry (MS) methods, chromatographic methods followed by MS, electrophoretic methods, electrophoretic methods followed by MS, nuclear magnetic resonance (NMR) methods, and combinations thereof.

In some embodiments, the labeled glycans can be analyzed by chromatographic methods, including but not limited to, liquid chromatography (LC), high performance liquid chromatography (HPLC), ultra performance liquid chromatography (UPLC), thin layer chromatography (TLC), amide column chromatography, and combinations thereof

In some embodiments, the labeled glycans can be analyzed by mass spectrometry (MS) and related methods, including but not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof

In some embodiments, the labeled glycans can be analyzed by electrophoretic methods, including but not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof

In some embodiments, the labeled glycans can be analyzed by nuclear magnetic resonance (NMR) and related methods, including but not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof

In some embodiments, the methods described herein allow for detection of glycans that are present at low levels within a population of glycans. For example, the present methods allow for detection of glycan species that are present at levels less than 10%, less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.025%, or less than 0.01% within a population of glycans.

In some embodiments, the methods described herein allow for detection of particular linkages that are present at low levels within a population of glycans. For example, the present methods allow for detection of particular linkages that are present at levels less than 10%, less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.025%, or less than 0.01% within a population of glycans.

In some embodiments, the methods described herein allow for detection of relative levels of individual glycan species within a population of glycans. For example, the area under each peak of a liquid chromatograph can be measured and expressed as a percentage of the total. Such an analysis provides a relative percent amount of each glycan species within a population of glycans.

The methods will be more specifically illustrated with reference to the following examples. However, it should be understood that the methods are not limited by these examples in any manner.

### EXAMPLES

Examples 1-5 describe exemplary methods for labeling glycans with an aminated label according to the present disclosure. It is to be understood that any aminated label including any of those that are described herein (e.g., 2-aminopyridine (2AP), 2-aminobenzamide (2AB), 2-aminobenzoic acid (2AA), etc.) can be used in each of these methods. Similarly, it is to be understood that a variety of reducing agents including those that are described herein (e.g., borane-dimethylamine complex, sodium cyanoborohydride complex, etc.) can be used.

### Example 1

A glycan preparation is placed in a reaction vial and frozen using liquid nitrogen. The preparation is then dried through vacuum sublimation under reduced pressure. In a separate vial, a labeling solution is prepared by dissolving the aminated label in a mixture of methanol and acetic acid at a final concentration of 0.35M. The labeling solution is then used to resuspend the dried glycan preparation and the reaction mixture is placed at 90° C. for 20 minutes. The reaction mixture is then dried on a centrifugal evaporator at 45° C. and a 1.2M solution of the reducing agent in methanol and acetic acid is added. The resulting mixture is heated at 90° C. for 35 minutes. The mixture is then dried in a centrifugal evaporator and excess aminated label is removed by dialysis. Finally, the labeled glycan is freeze-dried on a speed-vac and stored at −20° C. The preparation is maintained in a substantially solid form throughout the evaporation (i.e., drying) phase of the freeze-drying process. In one experiment, this method was used to label a glycan preparation with 2-aminopyridine (2AP).

### Example 2

A glycan preparation is placed in a reaction vial and frozen using liquid nitrogen. The preparation is then dried through vacuum sublimation under reduced pressure. In a separate vial, a labeling solution is prepared by dissolving the aminated label and the reducing agent in a mixture of methanol and acetic acid at a final concentration of 0.35M and 1.2M, respectively. The labeling solution is then used to resuspend the dried glycan preparation and the reaction mixture is placed at 70° C. for 2 hours. The resulting mixture is then dried in a centrifugal evaporator and excess aminated label is removed by dialysis. Finally, the labeled glycan is freeze-dried on a speed-vac and stored at −20° C. The preparation is maintained in a substantially solid form throughout the evaporation (i.e., drying) phase of the freeze-drying process. In one experiment, this method was used to label a glycan preparation with 2-aminopyridine (2AP).

### Example 3

A glycan preparation is placed in a reaction vial and frozen using liquid nitrogen. The preparation is then dried through vacuum sublimation under reduced pressure. In a separate vial, a labeling solution is prepared by dissolving the aminated label and the reducing agent in a mixture of dimethylformamide (DMF) and acetic acid at a final concentration of 0.35M and 1.2M, respectively. The labeling solution is then used to resuspend the dried glycan preparation and the reaction mixture is placed at 65° C. for 3 hours. The resulting mixture is then dried in a centrifugal evaporator and excess aminated label is removed by paper chromatography on a Watmann 3 filter. Finally, the labeled glycan is eluted with water, freeze-dried on a speed-vac and stored at −20° C. The preparation is maintained in a substantially solid form throughout the evaporation (i.e., drying) phase of the freeze-drying process. In one experiment, this method was used to label a glycan preparation with 2-aminobenzamide (2AB). In another experiment, this method was used to label a glycan preparation with 2-aminobenzoic acid also called anthranilic acid (2AA).

### Example 4

A glycan preparation is placed in a reaction vial and frozen using liquid nitrogen. The preparation is then dried through vacuum sublimation under reduced pressure. In a separate vial, a labeling solution is prepared by dissolving the aminated label in a mixture of dimethylsulfoxide (DMSO) and acetic acid at a final concentration of 0.35 M. The labeling solution is then used to resuspend the dried glycan preparation and the reaction mixture is placed at 65° C. for 1 hour. The reaction mixture is then dried on a centrifugal evaporator at 45° C. and a 1.2 M solution of the reducing agent in dimethylsulfoxide (DMSO) and acetic acid is added. The resulting mixture is heated at 70° C. for 2 hours. The mixture is then dried in a centrifugal evaporator and excess aminated label is removed by paper chromatography or dialysis. Finally, the labeled glycan is freeze-dried on a speed-vac and stored at −20° C. The preparation is maintained in a substantially solid form throughout the evaporation (i.e., drying) phase of the freeze-drying process.

### Example 5

A glycan preparation is placed in a reaction vial and frozen using liquid nitrogen. The preparation is then dried through vacuum sublimation under reduced pressure. In a separate vial, a labeling solution is prepared by dissolving the aminated label in a mixture of dimethylformamide (DMF) and acetic acid at a final concentration of 0.35 M. The labeling solution is then used to resuspend the dried glycan preparation and the reaction mixture is placed at 65° C. for 1 hour. The reaction mixture is then dried on a centrifugal evaporator at 45° C. and a 1.2M solution of the reducing agent in dimethylformamide (DMF) and acetic acid is added. The resulting mixture is heated at 70° C. for 2 hours. The mixture is then dried in a centrifugal evaporator and excess aminated label is removed by paper chromatography or dialysis. Finally, the labeled glycan is freeze-dried on a speed-vac and stored at −20° C. The preparation is maintained in a substantially solid form throughout the evaporation (i.e., drying) phase of the freeze-drying process.

Example 6 describes experiments that were performed in order to demonstrate the improved stability of labeled glycans that have been processed according to the methods described herein.

### Example 6

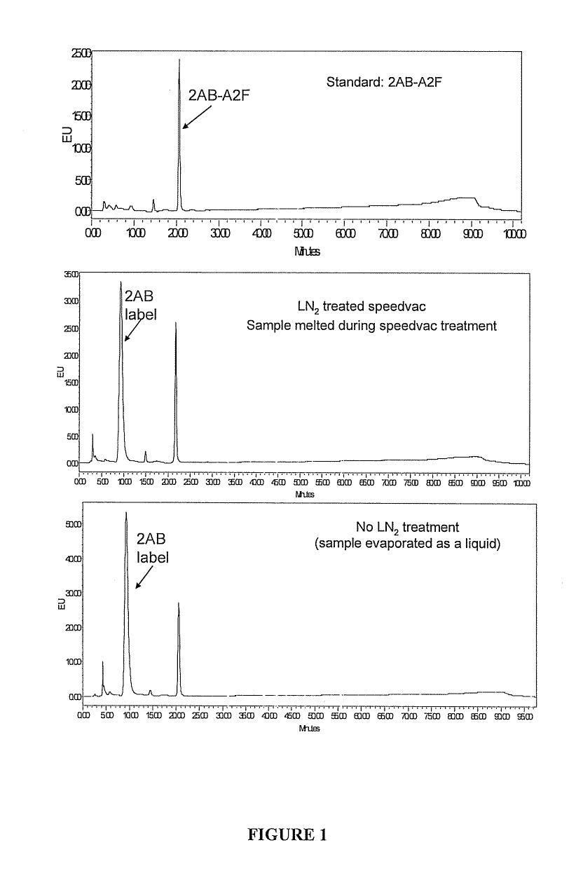

A 2AB-labeled glycan standard (2AB-A2F) was subjected to different types of post-labeling treatments. Preparation A was evaporated as a liquid. Preparation B was freeze-dried but the preparation was allowed to melt during speed-vac treatment. As shown in FIG. 1, HPLC analysis of these preparations showed that decomposition of the 2AB-A2F occurred if the preparation was allowed to evaporate as a liquid (Preparation A, FIG. 1, lower graph) or was allowed to melt during the evaporation process (Preparation B, FIG. 1, middle graph). For comparison, the spectrum of the unprocessed 2AB-A2F standard is also shown in FIG. 1 (upper graph). Decomposition typically resulted in elevated levels of the free 2AB label being detected by HPLC (retention time of ˜10 minutes as opposed to ˜21 minutes for the undecomposed 2AB-A2F).

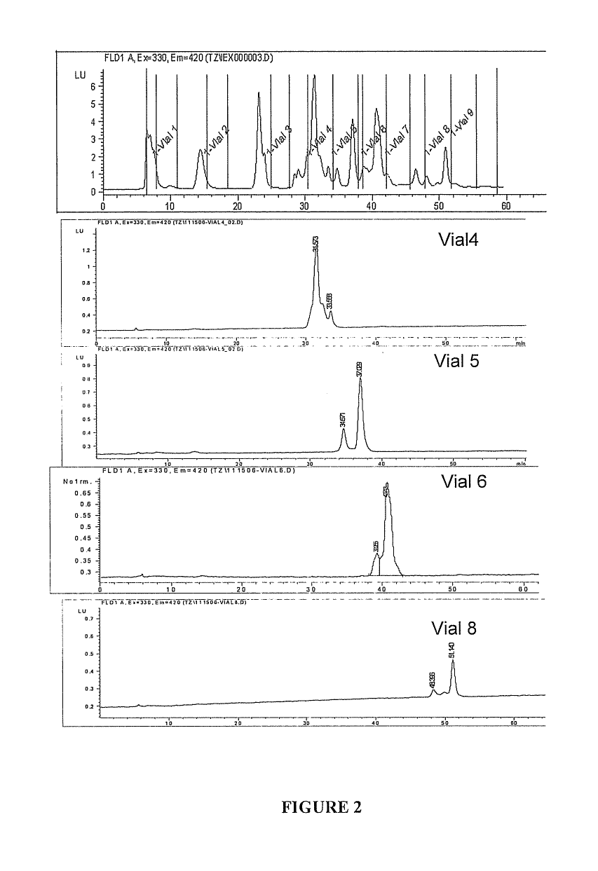

In contrast, when the preparation was maintained in a frozen state for the duration of the freeze-drying process, no decomposition was observed. This is illustrated in FIG. 2 for a mixture of 2AB-labeled glycans that were separated and fractionated by ion-exchange (IEX) chromatography (see FIG. 2, upper graph). Fractions 4, 5, 6 and 8 were collected and freeze-dried while maintaining the preparations in a substantially frozen state for the duration of the freeze-drying process. The fractions were then dissolved and analyzed by ion-exchange (IEX) chromatography. As shown in FIG. 2, no change in the elution profile, and no significant release of the free 2AB label, was detected demonstrating a significant improvement over the results for Preparations A and B (FIG. 1).

### Equivalents

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

The section headings used herein are for organizational purposes only and are not to be construed as limiting the subject matter described in any way.

While the methods have been described in conjunction with various embodiments and examples, it is not intended that the methods be limited to such embodiments or examples. On the contrary, the present disclosure encompasses various alternatives, modifications, and equivalents, as will be appreciated by those of skill in the art.

While the methods have been particularly shown and described with reference to specific illustrative embodiments, it should be understood that various changes in form and detail may be made without departing from the spirit and scope of the methods. Therefore, all embodiments that come within the scope and spirit of the present disclosure, and equivalents thereto, are intended to be claimed. The claims, descriptions and diagrams of the methods, systems, and assays of the present disclosure should not be read as limited to the described order of elements unless stated to that effect.

1. A method comprising steps of:
providing a labeled glycan preparation; and then
freeze-drying the labeled glycan preparation, wherein the labeled glycan preparation is maintained in a substantially frozen state for the duration of the freeze-drying step.

2. The method of claim 1, wherein the step of providing a labeled glycan preparation comprises:
providing a glycan preparation; and then
reacting the glycan preparation with an aminated label in the presence of a reducing agent so that the aminated label reacts with the glycan by reductive amination and becomes covalently linked to the glycan.

3. The method of claim 2, wherein the aminated label is a compound of formula I: 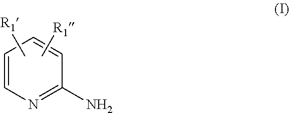
wherein
R₁′ and R₁″ are each independently H, NH₂, NHR₂, CONH₂, COOH, COR₃, COOR₄, SO₃, SOₙR₅ where n is 1 or 2, or an alkyl, alkoxyalkyl, alkenyl, alkynyl, cycloalkyl, cycloheteroalkyl, aryl or heteroaryl group, or when attached to adjacent carbon atoms R₁′ and R₁41 may be taken together with the atoms to which they are attached to form a 5- to 7-membered ring optionally containing a heteroatom selected from O, N or S; and
R₂, R₃, R₄ and R₅ are each independently H or an alkyl, alkoxyalkyl, alkenyl, alkynyl, cycloalkyl, cycloheteroalkyl, aryl or heteroaryl group.

4. The method of claim 2, wherein the aminated label is a compound of formula II: 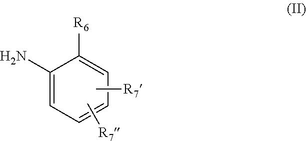
wherein
R₆ is NH₂, NHR₂, CONH₂, COOH, COR₃, COOR₄, SO₃ or SO₁R₅ where n is 1 or 2; and
R₇′ and R₇″ are each independently H, NH₂, NHR₂, CONH₂, COOH, COR₃, COOR₄, SO₃, SOₙR₅ where n is 1 or 2, or an alkyl, alkoxyalkyl, alkenyl, alkynyl, cycloalkyl, cycloheteroalkyl, aryl or heteroaryl group, or when attached to adjacent carbon atoms R₁ and R₁′ may be taken together with the atoms to which they are attached to form a 5- to 7-membered ring optionally containing a heteroatom selected from O, N or S; and
R₂, R₃, R₄ and R₅ are each independently H or an alkyl, alkoxyalkyl, alkenyl, alkynyl, cycloalkyl, cycloheteroalkyl, aryl or heteroaryl group.

5. The method of claim 2, wherein the aminated label is a compound of formula III: 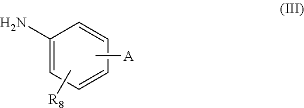
wherein
R₈ is NH₂, NHR₂, CONH₂, COOH, COR₃, COOR₄, SO₃ or SO₁R₅ where n is 1 or 2;
A is a fused 5- to 15-membered cycloheteroalkyl, aryl or heteroaryl ring system which is optionally substituted at 1 to 5 carbon positions with NH₂, NHR₂, CONH₂, COOH, COR₃, COOR₄, SO₃ or SOₘR₅ where m is 1 or 2, or an alkyl, alkoxyalkyl, alkenyl, alkynyl, cycloalkyl, cycloheteroalkyl, aryl or heteroaryl group; and
R₂, R₃, R₄ and R₅ are each independently H or an alkyl, alkoxyalkyl, alkenyl, alkynyl, cycloalkyl, cycloheteroalkyl, aryl or heteroaryl group.

6. The method of claim 5, wherein A is a fused 5- to 7-membered cycloheteroalkyl, aryl or heteroaryl ring system which is optionally substituted at 1 to 5 carbon positions with NH₂, NHR₂, CONH₂, COOH, COR₃, COOR₄, SO₃ or SOₘR₅ where m is 1 or 2.

7. The method of claim 5, wherein A is a fused 6-membered heteroaryl ring system which is optionally substituted at 1 to 5 carbon positions with NH₂, NHR₂, CONH₂, COOH, COR₃, COOR₄, SO₃ or SOₘR₅ where m is 1 or 2.

8. The method of claim 2, wherein the aminated label is selected from the group consisting of 2-aminopyridine, 2,6-diaminopyridine, 2-aminobenzoic acid, 2-aminobenzamide, ortho-phenylenediamine, 6-aminoquinoline, 8-aminonaphthalene-1,3,6-trisulfonic acid and 1,2-diamino-4,5-methylenedioxy-benzene.

9-15. (canceled)

16. The method of claim 2, wherein the reducing agent is selected from a borane dimethylamine complex and a sodium cyanoborohydride complex.

17. (canceled)

18. The method of claim 2, wherein the step of reacting is performed in a solution selected from the group consisting of:
(i) a solution that includes a mixture of methanol and acetic or citric acid;
(ii) a solution that includes a mixture of dimethylformamide and acetic or citric acid; and
iii) a solution that includes a mixture of dimethylsulfoxide and acetic or citric acid.

19-20. (canceled)

21. The method of claim 1, wherein the step of providing a labeled glycan preparation comprises:
providing a glycan preparation that includes a glycan with a sialic acid group; and then
reacting the glycan preparation with an aminated label so that the aminated label reacts with the sialic acid group via a condensation mechanism and becomes covalently linked to the glycan, wherein the aminated label is a compound of formula IIA: 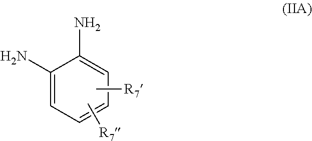
wherein
R₇′ and R₇″ are as defined in claim 4.

22. The method of claim 2, wherein in the step of providing a glycan preparation, the glycan preparation is a freeze-dried glycan preparation.

23. The method of claim 2, wherein in the step of providing a glycan preparation, the glycan preparation is a freeze-dried glycan preparation and the step of reacting comprises steps of:
re-suspending the freeze-dried glycan preparation by adding a solution that includes the aminated label; and then
adding a solution that includes the reducing agent.

24. (canceled)

25. The method of claim 2, wherein in the step of providing a glycan preparation, the glycan preparation is a freeze-dried glycan preparation and the step of reacting comprises steps of:
re-suspending the freeze-dried glycan preparation by adding a solution that includes the aminated label and the reducing agent.

26. (canceled)

27. The method of claim 2 further comprising a step of:
removing excess aminated label from the labeled glycan preparation before the freeze-drying step, wherein excess aminated label may be removed by paper chromatography or by dialysis.

28-29. (canceled)

30. The method of claim 27 further comprising a step of:
drying the labeled glycan preparation by evaporation before the step of removing.

31. (canceled)

32. The method of claim 1, wherein the step of freeze-drying comprises steps of:
placing the labeled glycan preparation in a container;
freezing the labeled glycan preparation by reducing the temperature within the container to below the eutectic point of the labeled glycan preparation; and
drying the labeled glycan preparation by reducing the pressure within the container.

33-35. (canceled)

36. The method of claim 32, wherein in the step of freezing, the temperature is reduced to a temperature in the range of about −240 to 0° C.

37. (canceled)

38. The method of claim 32, wherein in the step of freezing, the temperature is gradually reduced over a period of about 5 to 20 minutes.

39. The method of claim 32, wherein in the step of freezing, the temperature is cycled up and down within a temperature range.

40. The method of claim 39, wherein in the step of freezing, the temperature is cycled around a gradually decreasing temperature.

41. (canceled)

42. The method of claim 39, wherein in the step of freezing, the temperature is cycled anywhere within the range of about −240 to 25° C.

43. (canceled)

44. The method of claim 32, wherein in the step of drying, the pressure is reduced to a point where a solvent in the labeled glycan preparation can sublimate.

45-46. (canceled)

47. The method of claim 32, wherein in the step of drying, the temperature within the container is increased.

48-49. (canceled)

50. The method of claim 47, wherein in the step of drying, the temperature within the container remains at least 25° C. below the melting point of the labeled glycan preparation.

51. (canceled)

52. The method of any one of claims 3-5, wherein one or more of the following conditions is met:
(i) one or more of the hydrogen atoms is optionally isotopically labeled as ²H or ³H;
(ii) one or more of the carbon atoms is optionally isotopically labeled as ¹³C;
(iii) one or more of the oxygen atoms is optionally isotopically labeled as ¹⁸O;
(iv) one or more of the nitrogen atoms is optionally isotopically labeled as ¹⁵N; and
(v) one or more of the sulfur atoms is optionally isotopically labeled as ³³S or ³⁴S.

53-56. (canceled)
